(19) United States
(12) Patent Application Publication
(10) Pub. No.: US 2011/0262979 A1
(43) Pub. Date: Oct. 27, 2011

(54) NOVEL COMPOSITION OF MATTER FOR USE IN PRODUCING CO-ENZYME Q10 AND A NOVEL METHOD FOR PRODUCING CO-ENZYME Q10

(76) Inventors: Yangming Martin Lo, Ashton, MD (US); Yuting Tian, Fuzhou (CN)

(21) Appl. No.: 13/045,309

(22) Filed: Mar. 10, 2011

Related U.S. Application Data
(63) Continuation-in-part of application No. 12/864,416, filed on Nov. 2, 2010.
(60) Provisional application No. 61/312,604, filed on Mar. 10, 2010.
(60) Provisional application No. 61/023,515, filed on Jan. 25, 2008.
(60) Provisional application No. 61/106,426, filed on Oct. 17, 2008.

(51) Int. Cl. C12P 7/66; C12N 1/14; C12N 1/16; C12N 1/20

(52) U.S. Cl. 435/133; 435/253.6; 435/256.8; 435/255.7

(57) ABSTRACT

The present invention provides materials and methods for the growth of microorganisms for the production of organic chemicals, for example, coenzyme Q10.

### CROSS REFERENCE TO RELATED APPLICATIONS

This application is a Continuation-In-Part of copending application Ser. No. 864,416, filed Jan. 26, 2009, which is the U.S. National Stage of PCT/US2009/031995 which claims priority from the provisional application Ser. No. 61/023,515, filed Jan. 25, 2008 and 61/106,426 filed Oct. 17, 2008, the contents of which are hereby incorporated by reference in their entirety. This application also claims priority from the provisional application Ser. No. 61/312,604 which was filed Mar. 10, 2010, the entire contents of which are incorporated herein by reference.

### GOVERNMENT INTERESTS

This invention was made with U.S. government support under USDA-CSREES Awards No. 2008-34467-19445 and 2009-34467-20151. The government has certain rights in the invention.

### FIELD OF THE INVENTION

This invention relates to a novel composition of matter, plus a method for using this novel composition of matter to enhance the production of Co-Enzyme Q10.

### BACKGROUND OF THE INVENTION

U.S. patent publication 2011/0053224 discloses a novel composition of matter—leaf biomass hydrolysate (LBH). LBH can promote growth of microorganisms and can also be utilized in the production of organic chemicals, for example, organic acids or ethanol and other biofuels.

The present invention discloses a method for producing Co-Enzyme Q10 using LBH obtained from tobacco (Tobacco Leaf Biomass Hydrolysate or TLBH). Coenzyme Q₁₀ (CoQ10, also known as ubiquinone) is widely accepted as a potent antioxidative dietary supplement often linked with energy boosting, immune enhancement, and ease of hypertension (Ernster and Dallner, 1995; Sohet et al., 2009). Extensive attempts have been made to increase the production of CoQ10 to meet the growing demands.

Production of CoQ10 has generally followed one of the three routes: extraction from animal tissues (Serge et al., 2009), chemical synthesis (Ehud and Doron, 1988) or microbial fermentation (Yoshida et al., 1998). CoQ10 produced by fermentation is considered the most viable approach because of the ability to produce biologically potent CoQ10 without optical isomers and at reduced costs (Cluis et al., 2007; Choi et al., 2005a). Nonetheless, even fermentation-based production has been hindered by high costs and low yields (Matsuda et al., 2008; Matsuda et al., 2004).

There is some precedent for incorporating CoQ10 precursors into a growth medium to enhance CoQ10 production. Bule and Singhal (2009) found that carrot juice and tomato juice enhanced production of CoQ10 by Pseudomonas diminuta NCIM 2865 (Bule and Singhal, 2009). In the case of pure carrot juice, the authors reported nearly a 90% increase in CoQ10 production into an optimized fermentation growth medium after 96 hours. The cell growth in these modified media was not fully characterized, leaving considerable discrepancies in understanding where such stimulation effects took place and how they worked.

There remains a need in the art for improved materials and methods for the production of CoQ10.

### SUMMARY OF THE INVENTION

The present inventors have found that inclusion of TLBH in a growth medium resulted in a unexpectedly large increase in CoQ10 production—more than a 100% increase over the concentration produced by a growth medium which does not contain TLBH. (See FIG. 1). These findings indicate that inclusion of TLBH in growth media used to produce CoQ10 can dramatically increase production and reduce costs of CoQ10 production, and that a CoQ10 growth medium which contains TLBH can be a potent producer of CoQ10. Without wishing to be bound by theory, the authors believe that this increase may be due in part to the possible presence of solanesol, a CoQ10 precursor, in tobacco biomass. Solanesol has been reported to be present in tobacco leaf biomass (Machado et al., 2009), although it is not known whether TLBH itself actually contains solanesol. The present invention discloses a novel composition of matter consisting of (1) a microbial species suitable for the production of CoQ10; and (2) a culturing or growth medium for such species which contains TLBH. The present invention also discloses a method of producing CoQ10 utilizing this novel composition of matter.

Use of the present invention is capable of substantially increasing production of CoQ10 via fermentation compared with standard growth media. The inventors obtained more than a doubling of CoQ10 production compared with a standard commercial growth medium, using the present invention. Without being bound by theory, the inventors hypothesize that this dramatic increase in production is due to the combination of the microbial growth-promoting components of TLBH combined with the presence of solanesol, which is a precursor to CoQ10.

### BRIEF DESCRIPTION OF THE FIGURES

FIG. 1 Effect of supplemental TLBH on DCW and CoQ10 production by Rhodospirillum rubrum ATCC 25852 with batch culture.

FIG. 2. Typical growth profiles of R. rubrum ATCC 25852 cells in the optimized medium, or the optimized medium supplemented with 20% (v/v) TLBH, Alfalfa Leaf Biomass Hydrolysate (ALBH), or Spinach Leaf Biomass Hydrolysate (SLBH).

FIG. 3. CoQ10 production profiles of R. rubrum ATCC 25852 cells in the optimized medium, or the optimized medium supplemented with 20% (v/v) TLBH, ALBH, or SLBH.

FIG. 4. Effect of TLBH, ALBH, or SLBH on the specific CoQ10 content. Data are shown as mean±SD (n=3). Means with the same letter are not significantly different (P<0.05).

### DETAILED DESCRIPTION OF THE INVENTION

The principles, preferred embodiments and modes of operation of the present invention will be described hereunder. The invention which is intended to be protected herein should not, however, be construed as limited to the particular forms disclosed, as these are to be regarded as illustrative rather than restrictive. Variations and changes may be made by those skilled in the art without departing from the spirit of the present invention. Accordingly, the examples, descriptions, and best mode of carrying out the invention given below should be considered exemplary in nature and not as limiting to the scope and spirit of the invention as set forth in the claims.

The objective of the claimed method is to increase production of CoQ10 through fermentation through the addition of TLBH into the fermentation medium. The claimed novel composition of matter is a fermentation medium containing TLBH which is useful for the enhanced production of CoQ10.

TLBH may be prepared according to the methods described in U.S. patent publication 2011/0053224 to Lo et al. or by any other method which may be familiar to practitioners of the art. One suitable method for preparing TLBH is as follows:

TLBH is produced from tobacco leaves. The leaves are preferably harvested during their green vegetative stage and then processed as described in this application and in U.S. patent publication 2011/0053224 promptly following harvest. Prompt processing minimizes breakdown of the leaf sugars and proteins. As an alternative, the leaves can be refrigerated or frozen until ready for processing. Cooling the leaves will slow or prevent breakdown of sugars and proteins.

Following harvest or storage (as applicable), typically the leaves may be initially disrupted, crushed, pressed or chopped into small pieces. Any commonly used process for disrupting or chopping the leaf is acceptable, including chopping, milling, grinding or crushing the leaves, pulping, maceration procedures, mechanical pressure, rollers or homogenizing, among other such procedures which one skilled in the art will recognize. In a preferred embodiment, the disrupted leaves are also pressed, squeezed or rolled. This pressing, squeezing or rolling removes a substantial portion of the water from the leaves, along with water-soluble proteins contained in that water.

Following leaf disruption, desirable leaf components of interest to the practitioner may be extracted from the disrupted leaf. Such desirable leaf components may include, without limitation, soluble leaf proteins, enzymes, recombinant proteins or secondary metabolites. For example, the practitioner may wish to extract soluble leaf proteins from the disrupted leaf. (Johal, 1982).

The leaves are preferably then dried to a water activity level of approximately 0.85 or below in order to prevent the growth of microbial contaminants which may be present on the leaves. The leaves can be dried using any suitable drying technology such as oven drying or tunnel drying, amongst other methods which one skilled in the art may recognize.

It is possible at this point in the TLBH preparation process for the practitioner to package or commercially provide sterilized and/or disrupted leaves for distribution to users or customers. The leaves may be packaged in filter paper or in any other container which permits soluble leaf nutrients to be extracted and filtered out into water or other solvent. Alternatively, the leaves may be ground into a powder prior to distribution. Or alternatively, the sterilized and disrupted leaves may be distributed to users in their leaf form. In the event that the practitioner elects to distribute the leaves to users or customers as described in this paragraph, then the user or customer would perform the remainder of the TLBH preparation steps described in the succeeding paragraphs.

Following the optional leaf disruption and sterilization and the removal of desirable leaf components, the leaves may then be incubated in a solvent suitable for extracting the soluble leaf components. Such solvents may include water, ethanol, acetone, etc, which one skilled in the art will recognize. The solvent (e.g., water) may be heated to between 60° C. and 90° C. in order to solubilize the constituents of the leaves. In a preferred embodiment, the leaves are heated at a temperature between 75° C. and 85° C. In one preferred embodiment, the water used will be boiled tap water to minimize the risk of microbial contamination. In an especially preferred embodiment, the water used should be distilled water to minimize risks of microbial contamination or impurities caused by minerals or heavy metals. Leaves should be heated in water for at least 15 minutes, but may be heated for as long as a week or more. In an especially preferred embodiment, leaves should be heated for between 30 minutes and two (2) hours. This incubation treatment produces a liquid containing soluble compounds. If the incubation treatment was performed in water, the liquid will contain water-soluble compounds. In a preferred embodiment, the ratio of leaves to hot water should be about 10 grams of leaves to between 100 and 300 ml. of water.

The hydrolysate may then be filtered using any suitable filtration equipment to remove solid material. Following filtration, the liquid containing the soluble compounds may be dried down to a concentrated liquid or powder. Exemplary drying techniques include spray drying to a powder product, or evaporation to produce a liquid concentrate, amongst many other drying/dehydration techniques which one skilled in the art will recognize.

The end product is a novel composition of matter, specifically a powder (if drying is performed) or liquid (if drying is not performed) containing the soluble constituents of the leaf biomass (excluding any desirable leaf components which were removed). In a preferred embodiment in which leaf disruption and sterilization were performed and distilled water was the incubation liquid, the end product is a liquid or powder consisting of the sterilized water-soluble constituents of the disrupted leaf biomass.

For purpose of this application, the terms “tobacco leaf biomass hydrolysate” or “TLBH” refers to a liquid or solid product which results from the process described in this detailed description of the Invention. References in this application to TLBH “in its liquid form” refer to the TLBH liquid product referred to in the preceding sentence.

Any microorganism(s) suitable for the fermentation-based production of CoQ10 can be utilized to practice the claimed invention. A non-limiting list of such microorganisms used in the production of CoQ10 include bacteria such as Pseudomonas spp., Rhodosprillum spp., Rhodobacter spp.; Agrobacterium spp., Paracoccus spp. and Escherichia coli; niykds such as Neurospora spp. and Aspergillus spp.) and yeasts such as Candida spp., Rhodotorula spp. and Saitoella.

Any “base” growth medium suitable for the production of CoQ10 can be utilized to practice the claimed invention. Practitioners of the art will recognize many such growth media. One non-limiting example of a fermentation medium which the inventors have found to be suitable for production of CoQ10 when using the bacterial species Rhodosprillum rubrum comprises (g/l): malic acid 2.5, yeast extract 1.29, (NH₄)₂SO₄ 1.34, MgSO₄.7H₂O 0.2, K₂HPO₄ 0.9, KH₂PO₄ 0.6, ferric citrate 0.08, and EDTA 0.02.

According to this invention, the microbial species is cultured in a “base” growth medium supplemented suitable for the production of CoQ10 that is supplemented with TLBH. Any base growth medium suitable for the production of CoQ10 is appropriate. Practitioners skilled in the art will recognize that many different formulations of base media are appropriate for CoQ10 production and that the optimal growth medium in a particular instance will depend on many variables including the microbial species utilized.

As noted above, the claimed method involves the addition of TLBH to a base growth media for CoQ10 production; the claimed novel composition of matter involves a growth medium for CoQ10 containing TLBH. The optimal proportion of the growth medium which should contain TLBH can be determined by the practitioner based on experimentation, and will vary based on the microbial species used, the other (non-TLBH) components of the growth medium used and the method of culturing the microbial species.

Without wishing to be bound by theory, the inventors believe that in a preferred embodiment, if TLBH is in its liquid form the growth medium will normally contain between 5% and 40% TLBH (v/v). In a particularly preferred embodiment of the invention, the authors believe that the growth medium will normally contain between 15 and 25% TLBH (v/v) if TLBH is in its liquid form.

Culturing of the microorganism in the TLBH-containing medium may be performed under a variety of conditions and using any suitable method for such microorganism known to practitioners in the art; such conditions will be chosen by the practitioner based on the particular microbial species used, base media employed, proportion of TLBH used, the particular conditions of fermentation employed, and on the methods used for obtaining or purifying a final product. A skilled practitioner would be aware of all such conditions, and would be able to determine optimal culturing conditions with a minimum of experimentation.

As a non-limiting example, the inventors have found that suitable conditions for culturing—R. rubrum ATCC 25852 cells in a 250-ml flask using the composition of matter of the claimed invention are as follows: actively growing R. rubrum are inoculated into a flask containing approximately 200 ml of the growth medium containing approximately 20% TLBH in its liquid form (v/v), which has been previously sterilized at approximately 121° C. for 15 min. The initial pH of the medium upon inoculation is maintained between 6 and 8 or more preferably at approximately 6.9. The culture is then statically incubated, for example, under a tungsten lamp at 35° C. for 96 h.

### EXAMPLES

### Example 1

### Effect of Different Levels of TLBH on Production of CoQ10

For purposes of these examples and the Figures, all references to TLBH concentrations as a proportion of a total volume of a mixture, solution or growth medium (i.e., “v/v”) refer to TLBH in its liquid form prepared as described in this paragraph. TLBH was prepared as follows: dried tobacco leaf samples (10.0 g) of low-alkaloid tobacco variety Nicotiana tabacum L. cv. MD-609LA were placed in a 250 ml beaker containing 150 ml Distilled water and heated in a water bath at 75-80° C. for 2 hours. The hydrolysates were filtered through a Whatman No. 4 filter paper (Whatman Inc., Florham Park, N.J.) and stored at −16° C. before further uses.

Freeze-dried R. rubrum ATCC 25852 culture (ATCC, Manassas, Va.) was hydrated with 9 ml sterilized water and inoculated into the ATCC medium, which contained (g/l): malic acid 2.5, yeast extract 1, (NH₄)₂SO₄ 1.25, MgSO₄.7H₂O 0.2, CaCl₂.2H₂O 0.07, K₂HPO₄ 0.9, KH2PO₄ 0.6, ferric citrate 0.01, and EDTA 0.02. One milliliter trace element solution and 7.5 ml vitamin solution were also added to each liter of the ATCC medium. The trace element solution contained (g/l): ferric citrate 0.3, MnSO₄.H₂O 0.002, H₃BO₃ 0.001, CuSO₄.5H₂O 0.001, (NH₄)₆Mo₇O₂₄.4H₂O 0.002, ZnSO₄ 0.001, EDTA 0.05, and CaCl₂.2H₂O 0.02. The composition of vitamin solution was (g/l): nicotinic acid 0.2, nicotinamide 0.2, thiamine.HCl 0.4, and biotin 0.008. After incubation under a tungsten lamp (100 W, luminous flux=1130 lumens) at 35° C. for 48 h, the stock of R. rubrum was prepared by mixing the broth with sterilized glycerol (10% v/v) and stored at −70° C. until used.

The fermentation medium contained (g/l): malic acid 2.5, yeast extract 1.29, (NH₄)₂SO₄ 1.34, MgSO₄.7H₂O 0.2, K₂HPO₄ 0.9, KH₂PO₄ 0.6, ferric citrate 0.08, and EDTA 0.02.

Actively growing R. rubrum ATCC 25852 cells were inoculated into 250 ml Erlenmeyer flasks containing 200 ml medium that was previously sterilized at 121° C. for 15 min. The initial pH of the medium upon inoculation was maintained at 6.9. The culture was statically incubated under a tungsten lamp at 35° C. for 96 h.

The cell mass concentration was determined using a calibration curve correlating optical density at 620 nm and dry cell weight (DCW). The optical density at 620 nm was measured with a spectrophotometer (ThermoSpectronic, Rochester, N.Y.). The DCW was determined after the culture broth was centrifuged at 10,000 rpm (9159.4×g) for 30 min under 4° C. using a Beckman Coulter L7 Ultracentrifuge (Beckman Coulter, Inc., Fullerton, Calif.) equipped with a Type 70.1 Ti rotor to precipitate the suspended cells.

For assaying the CoQ10 content, the celLytic B (Sigma-Aldrich Co., St. Louis, Mo.) solution of 0.5 ml was added to the cell pellet acquired by the centrifugation procedures described above. After 30 min incubation, a solvent mixture of propanol and hexane (1:2 v/v) was added to the solution for cell lysis under vigorous mixing. The solvent phase, as well as that obtained by second extraction from the aqueous phase, were combined and evaporated to dry using a speed vacuum concentrator Rota-vapor (Heidolph, Heizbad, Laborota 4001, Germany). The dry residue was dissolved in ethanol and applied to a high-performance liquid chromatography (HPLC) system (LC-2010A, Shimadzu, Tokyo, Japan) with a μBondapak C18 (10 μm, 3.9×300 mm, Waters, Milford, Mass.) coupled to a UV detector (Waters 486). The column was eluted with ethanol and methanol (9:1, v/v) at a flow rate of 0.6 ml/min and a chromatogram was obtained by monitoring the absorbance at 275 nm. The CoQ10 content was identified and quantified by comparing the peak areas in the chromatogram with a calibration curve prepared by known concentrations of authentic CoQ10 standard (Sigma-Aldrich, St. Louis, Mo.).

All analyses were performed in triplicate. The experimental results obtained were expressed as means±SD. Statistical analysis was performed using the SPSS package (version 11.5, SPSS Inc., Chicago, Ill.). Data were analyzed by analysis of variance (p<0.05) and the means separated by Duncan's multiple range test.

To determine the effect of the TLBH on the growth of R. rubrum cells and the corresponding CoQ10 content recoverable from the culture, the fermentation medium was supplemented with various ratios of TLBH (5, 10, 15, 20, and 25% v/v).

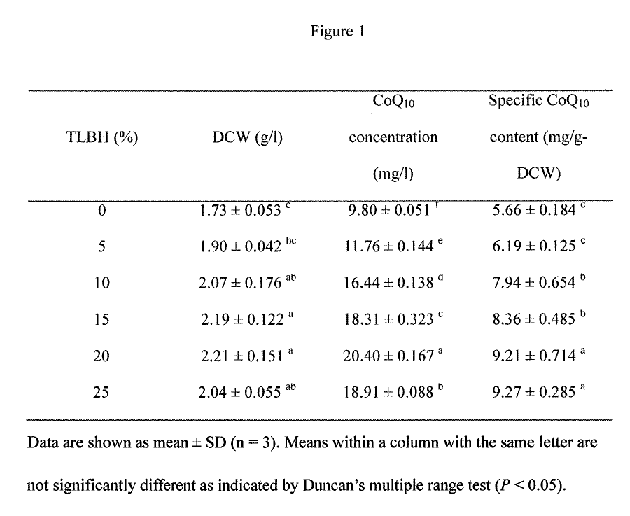

As shown in FIG. 1, CoQ10 concentration increased by more than 100% when 20% TLBH was added to the growth medium compared to a control. The CoQ10 concentration increased significantly with increasing TLBH supplementation ratio. The maximum CoQ10 concentration (20.4 mg/l) was reached in fermentation medium containing 20% TLBH.

The dried cell weight (DCW) of R. rubrum also increased with increasing TLBH supplementation up to 20%, and then decreased when the growth medium contained 25% TLBH.

The highest specific CoQ10 content, which was computed based on the quantity of CoQ10 accumulated per gram DCW, was found when the fermentation medium was supplemented with 20% or more TLBH, although the difference between 20% and 25% was insignificant.

Given the results described in this example, the optimum volumetric ratio of TLBH to this fermentation medium for CoQ10 production using R. rubrum ATCC 25852 culture was found to be 20% (v/v). This 20% concentration was therefore used for all subsequent experiments.

### Example 2

### Microbial Cell Growth in Association with CoQ10 Production in Different Growth Media

In addition to TLBH, biomass hydrolysates were prepared using alfalfa (ALBH) and spinach (SLBH), both according to the methods described in the first paragraph of Example 1 above (substituting alfalfa or spinach leaves, as applicable, for tobacco leaves) and cultured in the method set forth in Example 1 above. Alfalfa and spinach were selected because they are both potential sources of leaf protein, which may be produced as a co-product with leaf biomass hydrolysate. As in Example 1 above, all references to ALBH or SLBH concentrations (v/v) refer to ALBH or SLBH in their liquid form.

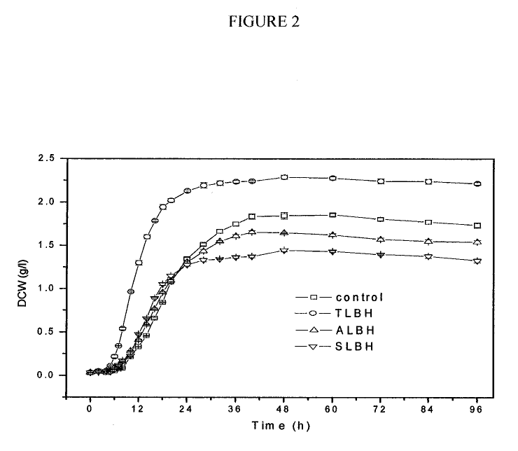

To characterize the growth of R. rubrum cells in various growth media, two growth-specific parameters were employed, namely the maximum biomass entering stationary phase (Xₘₐₓ) and the maximum specific growth rate (μₘₐₛₛ), an empirical parameter obtained from the steepest slope of the semi-logarithmic plot of cell density vs. growth time as defined by the Monod equation (Gardner et al., 2001). As can be seen in FIG. 2, R. rubrum grown in the medium supplemented with 20% TLBH showed the highest Xₘₐₓ (2.285 g/l) and μₘₐₓ (0.3584 h⁻¹), both significantly higher than those obtained in the other media (P<0.05). The cells grown in the broth containing 20% ALBH or SLBH did not show any significant differences in μₘₐₓ when compared with the control (the fermentation medium), which showed the Xₘₐₓ, significantly higher than those obtained in ALBH- and SLBH-supplemented media.

This result demonstrated that microbial growth in a fermentation medium of a species used to produce CoQ10 was substantially greater using TLBH than either a control or ALBH or SLBH, particularly during the first 24 hours.

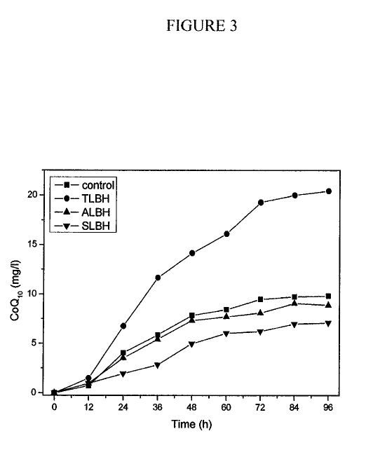

FIG. 3 shows the relative CoQ10 concentration on a volumetric basis of all fermentation broths investigated. At 36 hours into the fermentation, the amount of CoQ10 produced in the medium supplemented with 20% TLBH reached 12 mg/l, already exceeded the final CoQ10 concentration reached in the fermentation media (less than 10 mg/l) of those supplemented with ALBH or SLBH. The accumulation of CoQ10 in TLBH-supplemented medium reached more than 20 mg/l after 96 h of fermentation, more than twice those reached in the control or the other fermentation media.

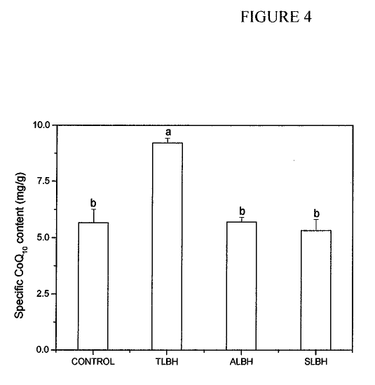

The specific CoQ10 content reached in fermentation supplemented with 20% TLBH (v/v) was also significantly higher than the other conditions investigated (FIG. 4), indicating that R. rubrum cells not only grew faster and reached higher cell density but also produced more CoQ10 per cell when TLBH was present in the growth medium.

### REFERENCES

U.S. Pat. No. 7,320,883, to Matsuda et al.
U.S. Pat. No. 6,762,037, to Matsuda, et al.
U.S. Pat. No. 4,334,024, to Johal, et al.
U.S. Pat. No. 3,769,170, to Kondo et al.
U.S. Patent Application 2010/0093054, to Lo et al.
WO/2009/094631, to Lo et al.
WO/2003/056024, to Yajima et al.
Bae, H. C., I. Cho, M. Nam (2005), “Effects of the biological function of yogurt added with Lycium chinence Miller extract.” Journal of Animal Science and Technology 47: 1051-1058.
Bule, M. and R. Singhal (2009), “Use of carrot juice and tomato juice as natural precursors for enhanced production of ubiquinone-10 by Pseudomonas diminuta NCIM 2865.” Food Chemistry 116: 302-305.
Choi, J., Y. Ryu, and J. Seo (2005a), “Biotechnological production and applications of coenzyme Q10.” Applied Microbiology Biotechnology 68: 9-15.
Choi, G., Y. Kima, J. Seo, and Y. Ryu (2005b), “Restricted electron flux increases coenzyme Q10 production in Agrobacterium tumefaciens ATCC4452.” Process Biochemistry 40: 3225-3229.
Cluis, C., A. Buria and V. Martin (2007), “Current prospects for the production of coenzyme Q10 in microbes.” Trends in Biotechnology: 25:514-521.
Ehud, I. and E. Doron (1988), “Total synthesis of polyprenoid natural products via Pd(0)-catalyzed oligomerizations.” Pure and Applied Chemistry: 60: 89-98.
Ernster, L. and G. Dallner (1995), “Biochemical, physiological and medical aspects of ubiquinone function.” Biochim Biophys Acta: 1271: 195-204.
Fu, H., P. Machado, T. Hahm, R. Kratochvil, C. Wei and Y. Lo (2010), “Recovery of nicotine-free proteins from tobacco leaves using phosphate buffer system under controlled conditions.” Bioresource Technology 101: 2034-2042.
Grammel, H. and R. Ghosh (2008), “Redox-state dynamics of ubiquinone-10 imply cooperative regulation of photosynthetic membrane expression in Rhodospirillum rubrum.” J. Bacteriol. 190: 4912-4921.
Gardner, N., T. Savard, P. Obermeier, G. Caldwell and C. Champagne (2001), “Selection and characterization of mixed starter cultures for lactic acid fermentation of carrot, cabbage, beet and onion vegetable mixtures.” Int. J. Food Microbiol. 64: 261-275.
Ha, S., S. Kim, J. Seo, H. Moon, K. Lee, J. Lee, (2007), “Controlling the sucrose concentration increases Coenzyme Q10 production in fed-batch culture of Agrobacterium tumefaciens.” Appl. Microbiol. Biotechnol. 76: 109-116.
Ha, S., S. Kim, J. Seo, D. Oh, J. Lee, (2007), “Optimization of culture conditions and scale-up to pilot and plant scales for coenzyme Q10 production by Agrobacterium tumefaciens.” Applied Microbiology and Biotechnology 74: 974-980.
Hamamura, K., I. Yamatsu, N. Minami, Y. Yamagishi, Y. Inai, S. Kijima, T. Namamura (2002), “Synthesis of [3′-14C] coenzyme Q10.” Journal of Label Compounds and Radiopharmaceuticals 45: 823-829.
Jeya, M., J. Moon, J. Lee, I. Kim, and J. Lee, “Current state of coenzyme Q(10) production and its applications (2010). Applied Microbiol Biotech. Feb 85: 1653-1663.
Jiang, S., L. Yu, X. Xiong, X. Shen and Y. Jian (2008), “Optimal supply of precursors for CoQ10 production by Rhodopseudomonas palustris.” Progress in Modern Biomedicine 8: 845-850.
Laplante, S., N. Souchet and P. Bryl (2009), “Comparison of low-temperature processes for oil and coenzyme Q10 extraction from mackerel and herring.” Eur. J. Lipid Sci. Technol. 111: 135-141.
Machado, P. A., H. Fu, R. Kratochvil, Y. Yuan, T. Hahm, C. Sabliov, C. Wei and Y. Lo (2010), “Recovery of solanesol from tobacco as a value-added byproduct for alternative applications.” Bioresour. Technol. 101: 1091-1096.
Naruta, Y. (1980), “Regio- and stereoselective synthesis of coenzyme Qn (n=2-10), vitamin K, and related polyprenylquinones.” J. Org. Chem. 45: 4097-4104.
Nishiyama, R., (1965), “Studies on components of Kuko (Lycium chinense Miller). Part III. Growth effect for lactic acid bacteria by the components of Kuko (Lycium chinense Miller).” Journal of the Agriculture Chemical Society of Japan 12: 313-319.
Oelze, J., W. Pahlke, S. Bohm (1975), “Ubiquinone 10 formation in Rhodospirillum rubrum under different culture conditions.” Archives of Microbiology 102: 65-69.
Sasaki, K., M. Watanabe, Y. Suda, A. Ishizuka, N. Noparatnaraporn (2005), “Applications of photosynthetic bacteria for medical fields.” Journal of Bioscience and Bioengineering 100: 481-488.
Sohet, F., A. Neyrinck, B. Pachikian, F. de Backer, L. Bindels, P. Niklowitz, T. Menke, P. Cani and N. Delzenne (2009), “Coenzyme Q10 supplementation lowers hepatic oxidative stress and inflammation associated with diet-induced obesity in mice.” Biochem. Pharmacol. 78: 1391-1400.
Wu, Z., G. Du, J. Chen (2003), “Effects of dissolved oxygen concentration and DO-stat feeding strategy on CoQ10 production with Rhizobium radiobacter.” World Journal of Microbiology and Biotechnology 19: 925-928.
Yeh, Y., T. Hahm, C. Sabliov, Y. Lo (2008), “Effects of Chinese wolfberry (Lycium chinense P. Mill.) leaf hydrolysates on the growth of Pediococcus acidilactici.” Bioresour. Technol. 99: 1383-1393.
Yen, H. W., T. Shih (2007), “The influences of aerobic-dark and anaerobic-light cultivation on CoQ10 production by Rhodobacter sphaeroides in the submerged fermenter.” Enzyme and Microbial Technology 41: 600-604.
Yen, H. and T. Shih (2009), “Coenzyme Q10 production by Rhodobacter sphaeroides in stirred tank and in airlift bioreactor.” Bioprocess Biosyst. Eng. 32: 711-716.
Yoshida, H., Y. Kotani and K. Ochiai (1998), “Production of ubiquinone-10 using bacteria.” J. Gen. Appl, Microbiol. 44: 19-26.
Yossa, I. (2008), “Stimulating effect of tobacco biomass hydrolysates on the growth of probiotic cells.” Poster Session 176-08, presented at the Institute of Food Technologists Annual Meeting+Food Expo, Jul. 1, 2008.
Zahiri, H., S. Yoon, J. Keasling, S. Lee, S. Kim, S. Yoon, Y. Shina (2008), “Coenzyme Q10 production in recombinant Escherichia coli strains engineered with a heterologous decaprenyl diphosphate synthase gene and foreign mevalonate pathway.” Metab. Eng. 8: 406-416.
Zhang, D., B. Shrestha, Z. Li, T. Tan, T. (2007), “Ubiquinone-10 Production Using Agrobacterium tumefaciens dps Gene in Escherichia coli by Coexpression System.” Mole. Biotechnol. 35: 1-14.

What is claimed is:

1. A composition of matter consisting of Tobacco Biomass Leaf Hydrolysate (TLBH) and a growth medium suitable for the production of CoQ10.

2. The composition of claim 1 wherein TLBH, if in its liquid form, constitutes between 5% and 40% (v/v) of the composition.

3. The composition of claim 1 wherein TLBH, if in its liquid form, constitutes between 15% and 30% (v/v) of the composition.

4. A method for producing CoQ10 comprising the following steps:
(A) preparing a growth medium suitable for the production of CoQ10;
(B) adding TLBH into the growth medium;
(C) adding a microorganism suitable for the production of CoQ10 into the medium to form a culture; and
(D) incubating the culture.

5. The method of claim 4 wherein in step (B) TLBH, if in its liquid form, constitutes at least 10% but no more than 40% (v/v) of the growth medium.

6. The method of claim 4 wherein in step (B), TLBH, if in its liquid form, constitutes between 15% and 30% (v/v) of the growth medium.

7. The method of claim 4 wherein in step (C) the microorganism in comprises at least one of Pseudomonas spp.; Escherichia coli; Rhodospirillum spp. Pseudomonas spp., Agrobacterium spp.; Paracoccus spp.; Neurospora; Aspergillus spp.; Candida spp.; Rhodotorula spp. and Saitoella spp.
